(19) United States
(12) Patent Application Publication
(10) Pub. No.: US 2009/0062550 A1
(43) Pub. Date: Mar. 5, 2009

(54) Process for the Large Scale Production of Rizatriptan Benzoate

(75) Inventors: Purna Chandra Ray, Secunderabad (IN); Mohan Bandari, Secunderabad (IN); Mohammed Qadeeruddin, Secunderabad (IN); Seeta Ramanjaneyulu Gorantla, Secunderabad (IN)

Correspondence Address: KNOBBE MARTENS OLSON & BEAR LLP, 2040 MAIN STREET, FOURTEENTH FLOOR, IRVINE, CA, 92614, US

(73) Assignee: MATRIX LABORATORIES LTD, Secunderabad (IN)

(21) Appl. No.: 12/093,683

(22) PCT Filed: Nov. 14, 2006
(86) PCT No.: PCT/IN06/00450

§ 371 Date: May 14, 2008

(30) Foreign Application Priority Data
Nov. 14, 2005 (IN) 1657/CHE/2005

(51) Int. Cl. C07D 249/08

(52) U.S. Cl. 548/262.2

(57) ABSTRACT

The present invention provides a method for preparing pure Rizatriptan benzoate having purity more than 99.5% and dimer impurity less than 0.1% comprises, i) Condensation of 1,2,4-Triazole with 4-Nitro benzyl bromide to yield 1-(4-nitrophenyl)methyl-1,2,4-triazole ii) Reducing the 1-(4-nitrophenyl)methyl-1,2,4-triazole to give 1-(4-aminophenyl)methyl-1,2,4-triazole iii) Converting 1-(4-aminophenyl)methyl-1,2,4-triazole to 1-(4-hydrazinophenyl)methyl-1,2,4-triazole hydrochloride iv) Condensing the hydrazine derivative with 4-(Dimethylamino) butanal diethylacetal to get Rizatriptan and v) Salification of Rizatriptan to Rizatriptan benzoate.

The present invention relates to an improved process for the large scale production of Rizatriptan benzoate in high yield with dimer impurity less than 0.1% and purity more than 99.5% with out involving hazardous reactions or reagents

### BACKGROUND OF THE INVENTION

N,N-Dimethyl-2[5-(1,2,4-triazol-1-ylmethyl)-1H-indol-3-yl]ethylamine (Rizatriptan) has the formula as given below (Formula-I).

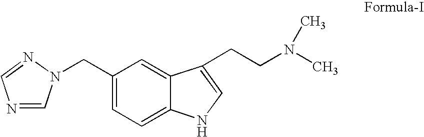

Rizatriptan and the physiologically acceptable salts are useful as 5-HT_{1B1D} receptor agonist, and is marketed as an oral formulation for acute treatment of migraine.

U.S. Pat. No. 5,298,520 and J. S. Leslie et al., J. Med. Chem., 1995, 38, and 1799 discloses Rizatriptan, methods for its preparation and pharmaceutical formulations using the same. The process disclosed in U.S. Pat. No. 5,298,520 involves the preparation of Rizatriptan by Fisher indole synthesis using the corresponding phenyl hydrazine and an aldehyde. The method described in that patent involves multi step synthesis accompanied by of column purifications. It also involves hazardous reagents such as NaH which results in the formation of more unwanted regioisomer of 1,2,4-Triazol-4-yl and stillbene derivative impurity. Again using this prior art process in Fisher indolization, which invariably results in the formation of a polar dimer impurity (Formula-II) to a large extent. Isolation of desired product without this dimer impurity is very cumbersome and expensive.

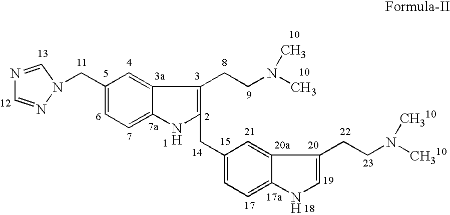

U.S. Pat. No. 5,567,819 discloses the preparation of 1-(4-hydrazinophenyl) methyl-1,2,4-triazole hydrochloride (Formula-III) which comprises reacting 4-amino-1,2,4-triazol with a nitrobenzene derivative containing a readily displaceable group. Subsequently conversion of this intermediate into Rizatriptan is carried out by Fisher indole synthesis. The end product nevertheless continues to have the disadvantages on quality of product even a column purification step is involved, so that it is not cost-effective to carry out the process in industrial scale.

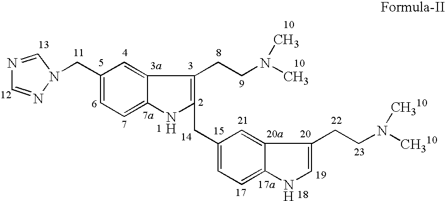

U.S. Pat. No. 5,567,824 also discloses a process for preparing Rizatriptan, by palladium-catalyzed coupling ring closure of 3-Iodine-4-aminobenzyl-triazol with a suitably protected butynol derivative to corresponding tryptophol followed by conversion of the hydroxyethyl moiety to dimethylaminoethyl moiety (Rizatriptan). Although this process does not require column purification, it has the disadvantage of using a palladium catalyst which makes the process more expensive, while also using highly toxic reagents such as iodine chloride and highly flammable reagents such as n-butyl lithium.

However, the above discussed processes have one major drawback in common in that the removal of polar dimer impurity of Formula-II using conventional techniques.

EP 0,925,302 discloses the preparation of 2-silyl protected indoles, by palladium-catalyzed cross-coupling reaction of halo anilines with acylsilanes, and preparation of product sought by deprotection of these intermediates so obtained.

U.S. Pat. Appl. No. 2005/0148778 discloses the synthesis of the Rizatriptan using hydrazine hydrochloride derivative of Formula-VI with α-keto-δ-valreolactone with low overall yield due to the lengthy process.

In the all above discussed prior art processes there are significant draw backs for commercial process with yield, quality and cost effective which are as.
Usage of hazardous, expensive, toxic and flammable reagents such as NaH, palladium, iodine chloride and n-butyl lithium.
Column chromatography, HPLC or other techniques thus to remove the unwanted regioisomeric impurities at early stages of the synthesis which make the commercial approach difficult.

Hence there is a need for a simple and commercially viable process for the preparation of Rizatriptan benzoate without involving the hazardous chemicals and it is also necessary to minimize the undesired regioisomer and stillbene derivative impurity at the early stages of the synthesis, which subsequently carried over to finished stage along with dimer impurity in the finished product.

Thus the present invention is provided with a commercially viable process which does not require hazardous & expensive catalyst like sodium hydride, palladium and the final product with dimer impurity less than 0.1%.

Unless other wise defined, all technical and synthetic terms used herein have the same ordinary meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods and examples are illustrative only and not intended to be limiting.

### SUMMARY OF THE INVENTION

The main object of the present invention is to provide an improved process for the preparation of Rizatriptan and its pharmaceutically acceptable salts.

Another object of the invention is to provide a process for preparation of Rizatriptan benzoate with purity more than 99.5%.

Another object of the invention is to provide a process for preparation of Rizatriptan benzoate with dimer impurity less than 0.1%.

Another object of the invention is to provide a process for preparation of Rizatriptan benzoate with out involving expensive hydrogenation catalysts or highly toxic and hazardous or highly flammable reagents.

Accordingly in the present invention Rizatriptan and its pharmaceutically acceptable salts are prepared by; i) Condensation of triazole with 4-nitro benzyl bromide to yield 1-(4-nitrophenyl) methyl-1,2,4-triazole ii) Reduction of 1-(4-nitrophenyl) methyl-1,2,4-triazole to 1-(4-aminophenyl) methyl-1,2,4-triazole iii) Converting 1-(4-nitrophenyl)methyl-1,2,4-triazole to 1-(4-hydrazinophenyl) methyl-1,2,4-triazole hydrochloride iv) condensing the hydrazine derivative with 4-(Dimethylamino) butanal diethylacetal to get Rizatriptan and v) Saltification of Rizatriptan to Rizatriptan benzoate.

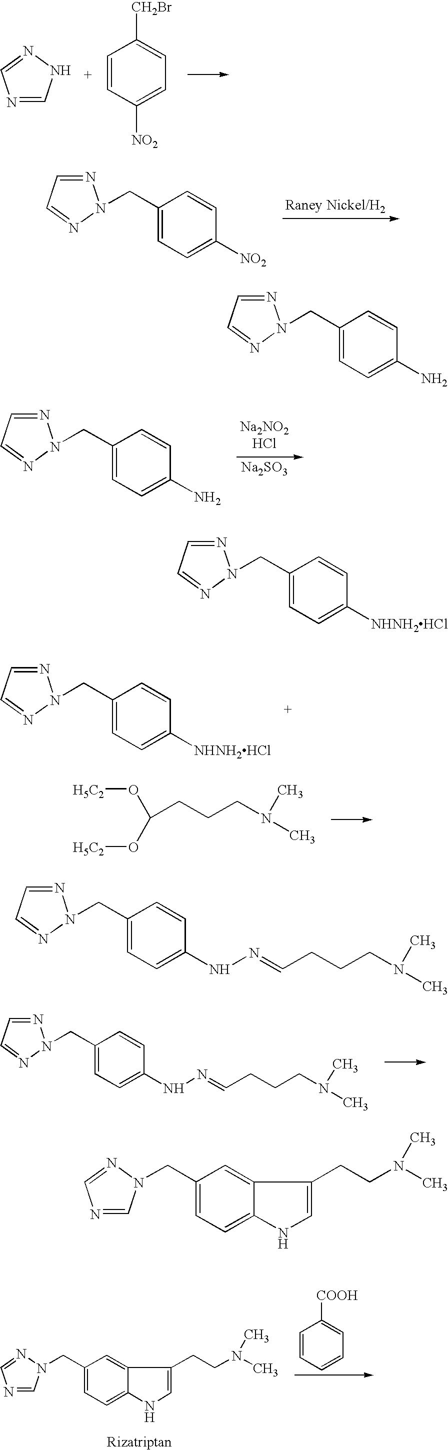

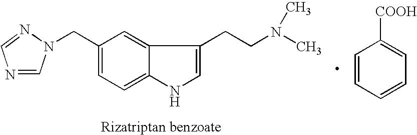

### DETAILED DESCRIPTION OF THE INVENTION

Thus in accordance with the present invention preparation of Rizatriptan benzoate comprises the following steps;
Condensation of triazole with 4-nitro benzyl bromide to yield 1-(4-nitrophenyl)methyl-1,2,4-triazole
Reduction of 1-(4-nitrophenyl) methyl-1,2,4-triazole to 1-(4-aminophenyl)methyl-1,2,4-triazole
Converting 1-(4-nitrophenyl) methyl-1,2,4-triazole to 1-(4-hydrazinophenyl)methyl-1,2,4-triazole hydrochloride
Condensing the hydrazine derivative with 4-(Dimethylamino)butanal diethylacetal to get Rizatriptan and
Saltification of Rizatriptan to Rizatriptan benzoate.

In a specific embodiment, the present invention provides a process for the preparation of Rizatriptan benzoate, which involves
i. Suspending 1,2,4-triazole in dipolar aprotic solvent selected from dimethyl formamide, dimethyl sulphoxide, sulpholane, N-Methyl-2-pyrrolidone and mixtures thereof wherein the preferable solvent is dimethyl formamide
ii. Adding an inorganic base selected from sodium carbonate, potassium carbonate, lithium carbonate or their corresponding bicarbonates
iii. Heating the mass to 90-95° C. and maintaining for about 5 hrs
iv. Cooling the mass to −10° C. to 20° C., preferably −5 to 15° C.,
v. Adding 4-nitro benzyl bromide at −5° C. to 25° C., preferably at 0 to 10° C.
vi. Maintaining the reaction mass at 20° C. to 40° C. preferably 25 to 30° C. for about 10 hr to 20 hrs, preferably 14 to 16 hrs
vii. Pouring the reaction mass into water below 25° C. and maintaining for about 2 to 3 hrs
viii. Isolating the 1-(4-nitrophenyl) methyl-1,2,4-triazole as a wet cake

Further reduction of the resulting 1-(4-nitrophenyl) methyl-1,2,4-triazole is carried out by
i. Suspending 1-(4-nitrophenyl) methyl-1,2,4-triazole, methanol and water in a hydrogenator
ii. Charging Raney-Nickel and hydrogenating the reaction mixture under a pressure of 4-8 kg preferably 5-6 kg at 25 to 60° C. preferably at 40 to 45° C.
iii. Filtering the catalyst and evaporating the solvent completely
iv. Charging IPA and isolating the 1-(4-aminophenyl) methyl-1,2,4-triazole

Conversion of the 1-(4-aminophenyl) methyl-1,2,4-triazole to 1-(4-hydrazino phenyl)methyl-1,2,4-triazole hydrochloride is carried out by
i. Dissolving 1-(4-aminophenyl) methyl-1,2,4-triazole in Conc. HCl
ii. Adding the above solution to sodium nitrite in water at −15° C. to 15° C., preferably at −15° C. to −10° C.
iii. Maintaining the reaction mixture at −15 to −10° C. for 30 min
iv. Raising the temperature to 25-30° C.
v. Charging HCl and maintaining for 2 hrs at 25-30° C.
vi. Isolating the product and washing with water followed by IPA to get 1-(4-hydrazinophenyl) methyl-1,2,4-triazole mono hydrochloride

Further condensation of 1-(4-hydrazinophenyl) methyl-1,2,4-triazole hydrochloride with 4-(Dimethylamino) butanal dimethylacetal is carried out by
i. Suspending 1-(4-Hydrazinophenyl)methyl-1,2,4-triazole hydrochloride in Conc.HCl and water
ii. Adding 4-(Dimethylamino) butanal diethylacetal at 20-25° C. and stirred for 60 min
iii. Heating the reaction mass to 70-75° C. and maintained for 60 min
iv. Cooling the mass to 30° C., adjusting the pH to 6.0-6.5
v. Extracting with methylene chloride
vi. Separating the aq. layer and adjusting the pH to 10-11 and extracting with ethyl acetate
vii. Removing ethyl acetate under vacuum
viii. Subjecting the residue to column chromatography and eluting with ethyl acetate followed by methanol to give Rizatriptan as residue.

The conversion of Rizatriptan to Rizatriptan benzoate is carried out by
i. Dissolving Rizatriptan residue in ethanol
ii. Adjusting the pH to 6.0-6.5 with benzoic acid in ethanol at 30-35° C.
iii. Cooling the mass to 0° C. and stir for 60 min
iv. Filtering the crude product and washing with ethanol
v. Dissolving crude Rizatriptan benzoate in ethanol at 78-80° C.
vi. Slowly cooling the mass to room temperature and allowing it to crystallize
vii. Finally cooling to 5-8° C. for 60 min.
viii. Isolating the product and washing with chilled ethanol to get Rizatriptan benzoate having purity more than 99.5% and dimer impurity less than 0.1%.

Methods known in the art may be used with the process of this invention to enhance any aspect of this process. For example the product obtained may be further purified by crystallization from solvent(s). The present invention is further illustrated by the following examples, which are provided nearly to the exemplary of the inventions and is not intended to limit the scope of invention. Certain modifications and equivalents will be apparent to those skilled in the art and are intended to be included with in the scope of the present invention in any way.

### EXAMPLE-1

### Preparation of 1-(4-Hydrazinophenyl)methyl-1,2,4-triazole hydrochloride

### Step-A: Preparation of 1-(4-nitrophenyl)methyl-1,2,4-triazole

1,2,4-Triazole (25 kg.) and potassium carbonate (53 kg.) are added to anhydrous DMF (125 lt.) at room temperature. The reaction mixture is heated to 90-95° C. and maintained for about 5 hrs. The reaction mixture is cooled to 0° C. and 4-nitro benzyl bromide (75 kg.) is added below 10° C. Reaction is maintained at 25-30° C. for 14-16 hrs. Finally the reaction mixture is poured into water (245 lt.) below 25° C. and maintained at 20-25° C. for 2-3 hrs. The product is filtered and slurry washed with water (100 lt) to yield 1-(4-nitrophenyl) methyl-1,2,4-triazole (49 kg)

### Step-B: Preparation of 1-(4-aminophenyl)methyl-1,2,4-triazole

1-(4-Nitrophenyl) methyl-1,2,4-triazole (35 kg.), methanol (350 lt.) and water (35 lt.) are suspended in a hydrogenator. To the suspension Raney Nickel (approx. 7.0 kg) is added and mixture is hydrogenated at 40-45° C. maintaining at 5-6 kg pressure. After the reaction completion, the catalyst is filtered through hyflo bed and washed the hyflo bed with methanol (15 lt.). Methanol is distilled off and IPA (35 lit.) is charged. Reaction mass is cooled to 20-25° C. and maintained at 20-25° C. for 1 hrs. The precipitated product is filtered and washed the product with IPA (17 lt.) to yield 1-(4-aminophenyl) methyl-1,2,4-triazole (25 kg)

### Step-C: Preparation of 1-(4-hydrazinophenyl)methyl-1,2,4-triazole hydrochloride

A solution of 1,4-aminophenyl methyl-1,2,4 triazole (23 kg.) in concentrated HCl (28 lt.) is added to sodium nitrite solution (10.6 kg. in 13 lt of water) in such a rate the temp. does not exceed −10° C. The reaction mixture is maintained for 60 min. at about −2° C. and added slowly to a precooled (−10° C.) sodium sulphite (41.4 kg.) and water (50 lt.) solution at 15° C. The reaction mixture is maintained at −10 to −15° C. for 30 min. Reaction mass is raised to 25-30° C. and HCl (35 lt.) is added at 25-30° C. and maintained for 2 hrs. The product is filtered, washed the product with water (90 lt.) followed by IPA (23 lt.) to yield 1-(4-hydrazinophenyl) methyl-1,2,4-triazole hydrochloride (33 Kg).

### EXAMPLE-2

### Preparation of Rizatriptan Benzoate

1-(4-Hydrazinophenyl)methyl-1,2,4-triazole hydrochloride (7.0 kg.) in Conc. HCl (35.0 lt.) is stirred for 30 min at 20-25° C., to that water (140.0 lt.) is added and stirred for another 30 min at 20-25° C. 4-(Dimethylamino) butanal diethylacetal (7.10 kg.) is added slowly at 20-25° C. and stirred for 60 min. The reaction mixture is hated to 70-75° C. and maintained for 60 min. After completion of reaction, cooled the mass to 30° C., adjusted the pH to 6.0-6.5 and extracted with methylene chloride (2×35 lt.). Aq. layer is separated and adjusted the pH to 10-11 and extracted with ethyl acetate (3×55 lt.). Ethyl acetate is removed under vacuum and the residue is chromatographed on silica gel (55 kg.) and eluted with ethyl acetate (25 lt.) followed by methanol (30 lt.) to give Rizatriptan as residue. The obtained residue is dissolved in ethanol (15 lt.) and pH is adjusted to 6.0-6.5 with benzoic acid (1.8 kg.) in ethanol (3.5 lt) at 30-35° C. The reaction mass is cooled to 0° C. and stirred for 1 hr. The product is centrifuged and washed with ethanol (3 lt) to yield Rizatriptan benzoate (3.0 kg.) with purity >99.0%.

### EXAMPLE-3

### Purification of Rizatriptan Benzoate

Crude Rizatriptan benzoate (7 Kg) obtained in example-2 is dissolved in ethanol (60 lt.) at 78-80° C. to give a clear colorless solution. The solution is slowly cooled to room temperature and allowed it to crystallize and finally cooled to 5-8° C. and stirred for 1 hr. The purified product is then centrifuged, washed with chilled ethanol (7 lt.) and dried to give 6.25 kg pure Rizatriptan benzoate with dimer impurity <0.1%.

### Dimer Impurity:

The mother liquors from the benzoate formation could be further processed by a combination of fractional crystallization and column chromatography to yield the dimer impurity of formula-II

¹H NMR [300 MHz ¹H NMR (DMSO-d₆, δ)]: 2.28 [S, 12H, 2×-N(CH₃)₂]; 2.35-2.90 (m, 8H, 4×-CH₂); 4.10 (s, 2H, —CH₂ at C-14); 5.41 (s, 2H, —CH₂ at C-11); 6.94-7.44 (m, 7H, Aromatic U); 7.93 (s, 1H, CH at C-13); 8.59 (s, 1H, —CH at C-12); 10.86 (s, 1H, —NH exchangeable) &10.72 (s, 1H, —NH exchangeable)

¹³C NMR [300 MHz ¹³C NMR (DMSO-d₆, δ)]: 21.49 (C-8) 22.50 (C-22); 32.06 (C-14); 44.43 (C-10); 53.15 (C-11); 59.29 (C-23); 59.46 (C-9); 107.79 (C-7); 110.82 (C-17); 111.23 (C-20); 111.64 (C-3); 117.63 (C-6); 117.74 (C-16); 120.47 (C-4); 121.81 (C-21); 122.84 (C-19); 125.77 (C-5); 127.26 (C-15); 128.05 (C-3a); 129.21 (C-20a); 134.90 (C-7a); 135.02 (C-17a); 136.91 (C-2); 143.63 (C-12) & 151.30 (C-13)

Mass (N+H): 470.3

We claim:

1. A process for the commercial production of Rizatriptan benzoate comprises of the following steps,
a) Condensation of 1,2,4-Triazole with 4-Nitro benzyl bromide to yield 1-(4-nitrophenyl)methyl-1,2,4-triazole in an dipolar aprotic solvent in presence of an inorganic base
b) Reducing the 1-(4-nitrophenyl)methyl-1,2,4-triazole to give 1-(4-aminophenyl)methyl-1,2,4-triazole
c) Converting 1-(4-aminophenyl)methyl-1,2,4-triazole to 1-(4-hydrazinophenyl)methyl-1,2,4-triazole hydrochloride
d) Condensing the hydrazine derivative with 4-(Dimethylamino)butanal diethylacetal to get Rizatriptan and
e) Converting Rizatriptan to Rizatriptan benzoate.

2. The process as claimed in claim 1, wherein the step a) dipolar aprotic solvent is selected from DMF, DMSO, sulpholane, N-Methyl-2-pyrrolidone and mixtures thereof

3. The process as claimed in claim 1, wherein the step a) inorganic base is selected from Sodium carbonate, potassium carbonate, Lithium carbonate or their corresponding bicarbonates.

4. The process as claimed in claim 1, wherein the step a) preferably takes place in DMF in presence of potassium carbonate.

5. The process as claimed in claim 1, wherein the addition of 4-Nitro benzyl bromide takes place at about −5° C. to about 25° C., preferably at 0° C. to 10° C.

6. The process as claimed in claim 1, wherein the step b) is carried out by hydrogenation

7. The process as claimed in claim 1, wherein the step b) reduction is effected using Raney-Nickel

8. The process as claimed in claim 1, wherein the step c) is carried out by
Reacting 1-(4-aminophenyl)methyl-1,2,4-triazole with sodium nitrite, and
Reducing the formed diazonium salt with sodium sulphite

9. The process as claimed in claim 1, wherein the step c) is carried out at −15° C. to +15° C., preferably at −15° C. to −10° C.

10. The process as claimed in claim 1, wherein the step c) the product is isolated as mono hydrochloride

11. The process as claimed in claim 1, wherein the step d) is carried out in an inorganic acid at a relatively high dilution and low temperature.

12. The process as claimed in claim 1, wherein the step d) the preferable inorganic acid is hydrochloric acid

13. The process as claimed in claim 1, wherein the step d) ring closure is carried out at a temperature of 40° C. to 100° C., preferably at 70° C. to 75° C.

14. The process as claimed in claim 1, wherein the step e) is carried out in ethanol

15. The process as claimed in claim 1, wherein the Rizatriptan benzoate obtained having purity more than 99.0%

16. The process as claimed in claim 15, wherein the Rizatriptan benzoate obtained having purity more than 99.5%

17. The process as claimed in claim 1, wherein the Rizatriptan benzoate obtained containing dimer impurity less than 0.5%, preferably less than 0.3% and most preferably less than 0.1%.

18. The process as claimed in claim 1, wherein the Rizatriptan benzoate obtained having purity more than 99.5% and dimer impurity less than 0.1%

19. Rizatriptan benzoate containing less than 0.5%, wt/wt, dimer impurity

20. Rizatriptan benzoate of claim 19, containing less than 0.3%, wt/wt, dimer impurity

21. Rizatriptan benzoate of claim 20, containing less than 0.1%, wt/wt, dimer impurity
